(19) United States
(12) Patent Application Publication
(10) Pub. No.: US 2007/0043322 A1
(43) Pub. Date: Feb. 22, 2007

(54) Disposable safety syringe structure

(75) Inventors: Shuei-Yuan Lee, Taipei City (TW); Pei-Hsin Pei, Sinjhuang City (TW); Tu-Sing Hsueh, Yonghe City (TW)

Correspondence Address: TROXELL LAW OFFICE PLLC, SUITE 1404, 5205 LEESBURG PIKE, FALLS CHURCH, VA, 22041, US

(73) Assignee: Acxing Industrial Co., Ltd.

(21) Appl. No.: 11/335,671

(22) Filed: Jan. 20, 2006

(30) Foreign Application Priority Data
Jul. 28, 2005 (TW) 094125508

(51) Int. Cl. A61M 5/00

(52) U.S. Cl. 604/110; 604/197

(57) ABSTRACT

A disposable safety syringe structure includes a barrel, a needle seat, a plunger, and a protective cover. A circumferentially surrounded thin broken portion is disposed on a periphery of an end portion of the barrel, and a circular groove is disposed on the end portion of the barrel thereby fastening the protective cover. In addition, a vertical cutter is disposed on a bottom end of the protective cover, and a fastener is disposed on a top end of the plunger, such that the syringe can be hooked onto an opening of the outlet of the barrel by means of the fastener of the plunger after injection. When the protective cover is turned, the cutter cuts off the broken portion such that the needle head is retracted to the barrel and the barrel can be completely destroyed in order to prevent it from being recycled and reused.

### BACKGROUND OF THE INVENTION

(a) Field of the Invention

The present invention relates to a disposable safety syringe structure, more particularly, to a syringe structure, wherein a barrel thereof is broken by cutting, and then a needle head is pulled back and retracted to a barrel after an injection has been made, so as to completely damage the barrel and thus prevent it from being recycled and reused.

(b) Description of the Prior Art

The needle head is used to position on the barrel after an injection has been made with conventional syringes. Therefore, during the process, the needle head is first separated from the barrel, and then both of them are destroyed separately. As the entire syringe structure is kept intact before the process of destroying, there is a possibility that some dishonest businessmen recycle and reuse it, thereby causing pollution and inflammation. Therefore, some providers have designed several retractable syringes, so that the needle head is retracted to the barrel after an injection has been made, in order to prevent it from being reused. For example, Taiwan Utility Model No. 88211100 (as shown in FIG. 1) illustrates a hook structure 111 is disposed on a needle head 11, and then a pull hook 121 is disposed on a plunger 12. After an injection has been made, the pull hook 121 is inserted into the hook structure 111, so as to retract the needle head 11. In addition, Taiwan Utility Model No. 92201914 (as shown in FIG. 2) illustrates a retractable needle head 21, and a fastening portion 221 is disposed on a front end of a plunger 22 corresponding to the needle head 21. The fastening portion 221 is hooked onto the needle head 21 after an injection has been made, and the needle head 21 is then retracted to a barrel therein. Referring to FIG. 3, Taiwan Utility Model No. 91216494 illustrates a retractable member 311 is disposed on a needle head 31, and a fastening element 321 is disposed at a front end of a plunger 32, which pulls back the needle head by hooking the fastening element 321 and the retractable member 311, after an injection has been made.

Regarding the above-mentioned utility model applications, no matter how the plunger and the needle head are combined and retracted, a needle seat therein is required to be completely tightened to the barrel, so as to prevent the injection solution from leaking improperly. In addition, effortlessness and easy operation are to be taken into account, when pulling back the needle head. Therefore, this involves tests required for the precision, pressure resistance, pull resistance, and retraction force of these two elements, thereby causing troubles during mass production, increasing the defective rate, and making it impossible to lower production costs. Therefore, it is difficult to apply the above-mentioned structures universally.

To overcome the above shortcomings, the applicant of the present application previously filed an application disclosing a retractable syringe (as shown in FIG. 4), wherein a needle head 104 and a needle seat. 105 are disposed on an end portion of an barrel 101, and then a screw hole 106 is disposed on an internal rim of a bottom of an outlet thereof, whereas a thin broken portion formed and configured by a continuously pointed groove 107 is disposed on a periphery of an end of the barrel 101. In addition, a straight-rod-shaped bolt 1031 is disposed on the central position of a top of the plunger 103 thereon.

By means of the above-mentioned structures, a retractable safety syringe is configured and formed, so the plunger 103 injects the injection solution from the barrel 101 into a patient's body. In addition, after the injection has been made, the bolt 1031 of the plunger 103 is locked on the screw hole 106 of the barrel 101 therein, and then the plunger 103 is pulled back to break the groove 107 formed on the broken portion of the barrel 101 due to the pull force, thereby making the needle seat 105 and the needle head 104 retract to an interior of the barrel 101. Therefore, this can prevent the syringe from being recycled and reused after it has been used, and thus preventing people from being infected by the needle head 104.

### SUMMARY OF THE INVENTION

A primary object of the present invention is to provide a disposable safety syringe structure, capable of facilitating the retraction of the needle head by cutting after it has been used for injection, and also facilitating easy production and enhancing air tightness, so as to lower the defective rate and production costs.

To achieve the above object of the present invention, the disposable safety syringe structure disclosed in the present invention comprises a barrel, a needle seat, a plunger, and a protective cover, wherein a tubular outlet is disposed on an end portion of the barrel, and an opening is formed on an internal edge of a bottom of the outlet; then a circumferentially surrounded thin broken portion is disposed on a periphery of an end thereof. A circular groove is disposed on the end portion adjacent to a surface of the barrel, in order to fasten and position the protective cover therein. In addition, a vertical cutter is disposed on a bottom rim of the protective cover, and then a fastener is disposed on a top end of the plunger, such that the syringe can be hooked onto the opening formed on the internal rim of the outlet of the barrel by means of the fastener of the plunger, after the syringe has been used for injection. When the protective cover is turned, the cutter cuts off the broken portion thereby enabling the needle head being retracted to the barrel preventing the syringe from being recycled and reused after it has been used for injection.

According to the above-mentioned syringe structure, a slanted step portion is disposed on a bottom rim of the fastener of the plunger, such that the fastener first penetrates into the opening at the end portion of the syringe, and then is tightly hooked onto a sheet, so as to facilitate the retraction of the plunger. In addition, the slanted step portion makes the retracted needle head tilt, thereby preventing the needle head from being pushed back, recycled and reused, and causing infection.

According to the above-mentioned syringe structure, the broken portion disposed on the barrel is configured into a circular groove in shape of thin sheet, thereby facilitating the cutting off of the broken portion by the cutter of the protective cover along the circular groove.

According to the above-mentioned syringe structure, an annular groove is circumferentially formed on the end portion adjacent to the surface of the barrel, with pores formed in the center, and the protective cover having a fastener on its side thereof is covered on the end portion, and then hooked onto the annular groove by the fastener, in order to position the protective cover.

To enable a further understanding of the objectives and the technological methods of the invention herein, the brief description of the drawings below is followed by the detailed description of the preferred embodiments.

### BRIEF DESCIPTION OF THE DRAWINGS

FIG. 1 shows a cross-sectional perspective view of a first conventional syringe.

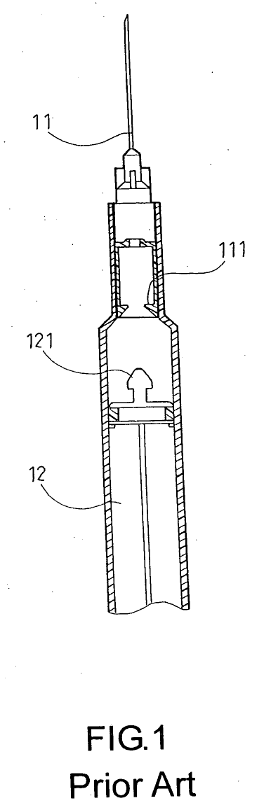

FIG. 2 shows a cross-sectional perspective view of a second conventional syringe.

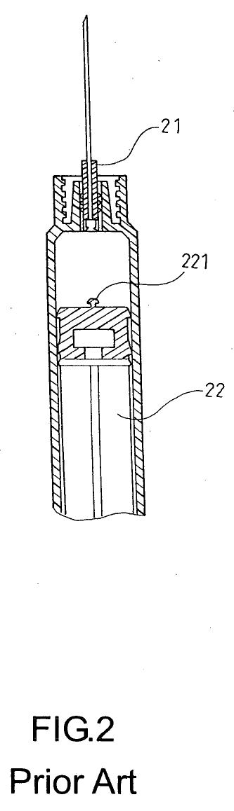

FIG. 3 shows a cross-sectional perspective view of a third conventional syringe.

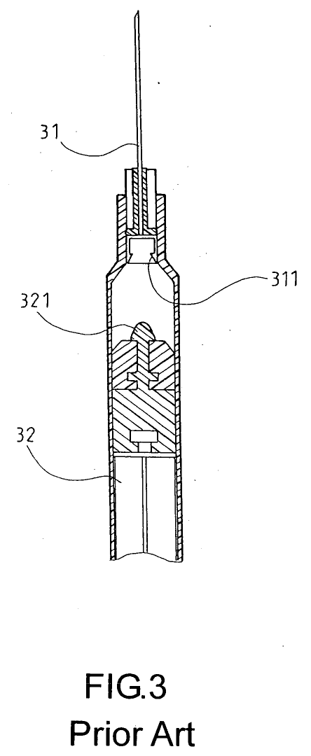

FIG. 4 shows a perspective exploded view of a conventional syringe.

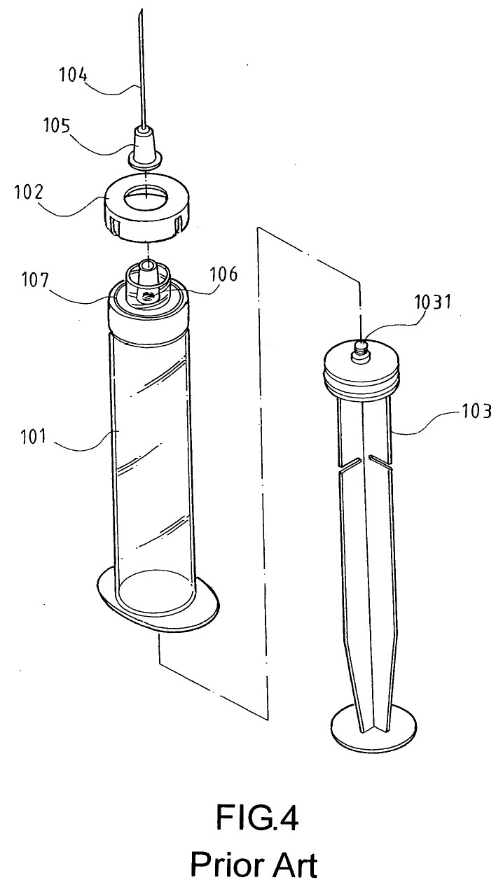

FIG. 5 shows a perspective view of the present invention.

FIG. 6 shows a perspective exploded view of the present invention.

FIG. 7 shows a perspective view of the protective cover according to the present invention.

FIG. 8 shows a plan view of the protective cover according to the present invention.

FIG. 9 shows a cross-sectional perspective view of the present invention.

FIG. 10 shows a cross-sectional perspective view applying to an embodiment of injection according to the present invention.

FIG. 11 shows a cross-sectional perspective view applying to an embodiment illustrating the hooking of the fastener and the opening according to the present invention.

FIG. 12 shows a cross-sectional perspective view applying to an embodiment of cutting according to the present invention.

FIG. 13 shows a cross-sectional perspective view applying to an embodiment of retraction according to the present invention.

FIG. 14 shows a cross-sectional perspective view applying to an embodiment illustrating the breaking of the plunger according to the present invention.

### DETAILED DESCRIPTION OF THE PREFERRED EMBODIMENTS

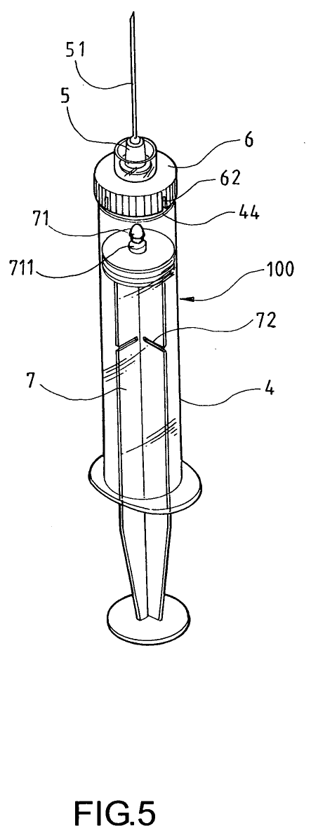

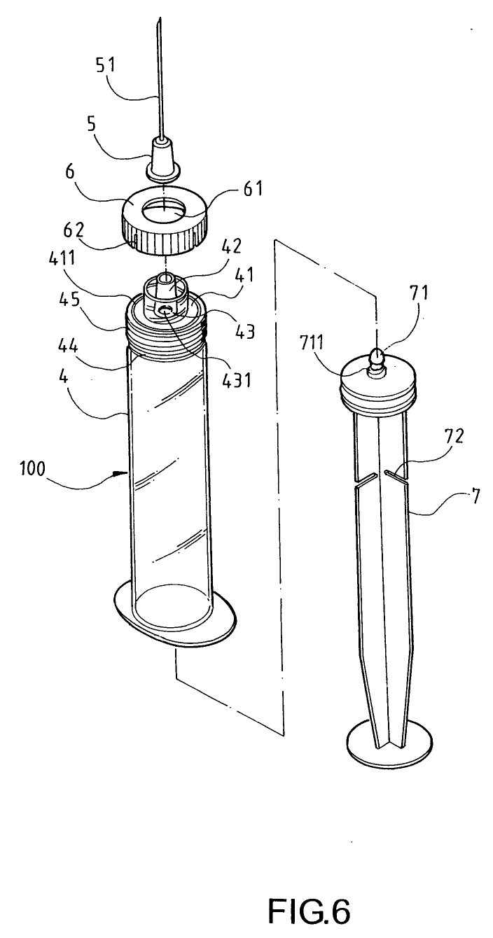

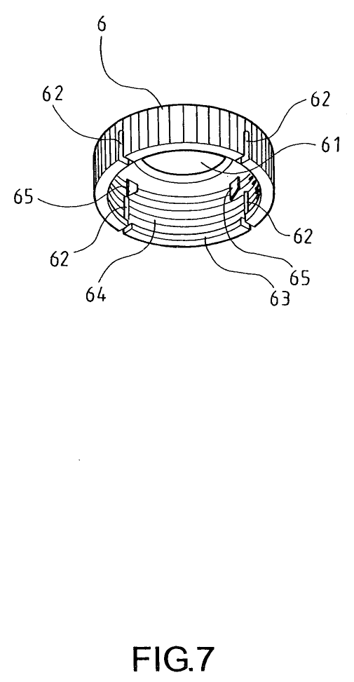

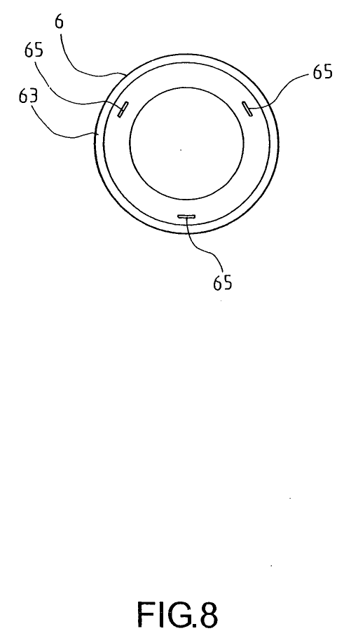

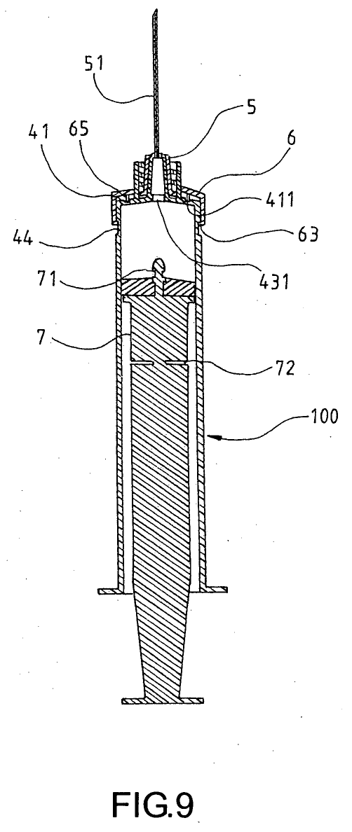

Referring to FIGS. 5, 6, 7, 8, and 9, the present invention comprises a barrel 4, a needle seat 5, a protective cover 6, and a plunger 7, wherein a tubular outlet 42 is disposed on an end portion 41 of the barrel 4 thereby fitting and positioning the needle seat 5 with a needle head 51. A sheet 43 is disposed on an internal rim at a bottom of the outlet 42, having an opening 431 formed on a central of the sheet 43, whereas a thin broken portion formed by a circular groove 411 is disposed on a periphery of an end portion 41 of the barrel 4. Moreover, an annular groove 44 is formed circumferentially on the end portion adjacent to a surface of the barrel 4, and a male screw thread 45 is formed on a top of the annular groove 44.

The protective cover 6 is in shape of a hat, wherein a pore 61 is disposed at a center thereof, a slot 62 is formed on a side thereof, a wedge fastener 63 and a female screw thread 64 are formed on an internal rim thereof. A vertical cutter 65 arranged in intervals corresponding to the circular groove 411 of the barrel 4 is disposed on an internal bottom of the protective cover 6. The protective cover 6 is turned onto the end portion 41 of the barrel 4 by the female screw thread 64, along the male screw thread 45 on the top of the annular groove 44 of the barrel 4. Therefore, the wedge fastener 63 at a bottom rim thereof can be hooked onto a top rim of the annular groove 44 on a surface of the barrel 4. The protective cover 6 is fastened by a few turns on a top of the end portion 41, thereby making a top of the cutter 65 clipping on a surface of the circular groove 41 1, further enabling needle seat 5 and the needle head 51 penetrating through the pore 61 of the protective cover 6.

The plunger 7 is provided with a vertical fastener 71 (in shape of a mushroom head in this embodiment) on a central position of a top thereof, and a slanted step portion 711 having a larger diameter is disposed on a bottom edge of the vertical fastener 71, whereas a rectangular hole 72 is disposed on a front rim of a handle of the plunger 7 thereof.

By means of the above-mentioned structures, a disposable safety syringe 100 is configured and formed. When it is used, the plunger 7 injects the injection solution from the barrel 4 into a patient's body. In addition, after the injection has been made, the vertical fastener 71 of the plunger 7 is hooked onto the screw hole 431 of the barrel 4 therein. Moreover, the protective cover 6 is turned and rotated downward, thereby enabling the cutter 65 cutting off the circular groove 411. Therefore, the plunger 7 is easily pulled back, so that the outlet 42 and the needle seat 5 together with the needle head 51 are retracted to the barrel 4 therein, in order to prevent the syringe 100 from being recycled and reused after it has been used, and prevent the needle head 51 from being infected by completely damaging the barrel.

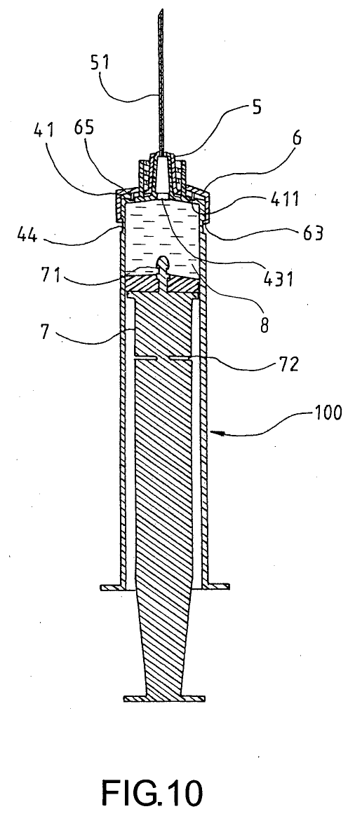

Referring to FIG. 10, when in use, the plunger 7 is pulled back, so as to inject the injection solution 8 into the barrel 4 therein. When making an injection, the plunger 7 is pushed forward to inject the injection solution 8 into the patient's body.

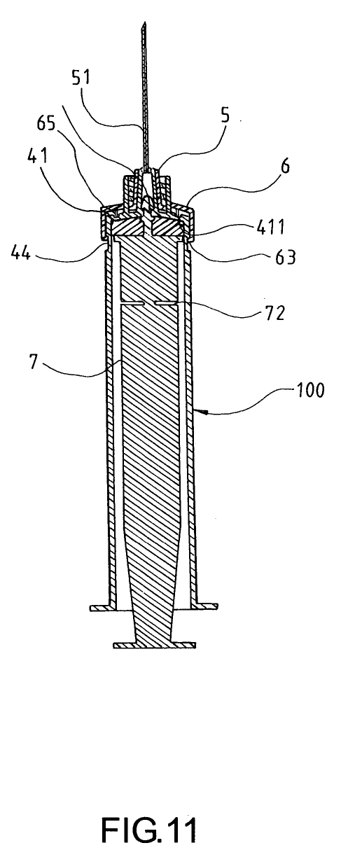

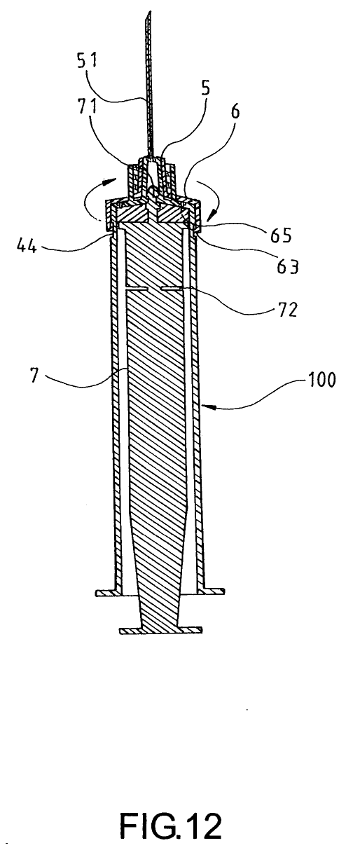

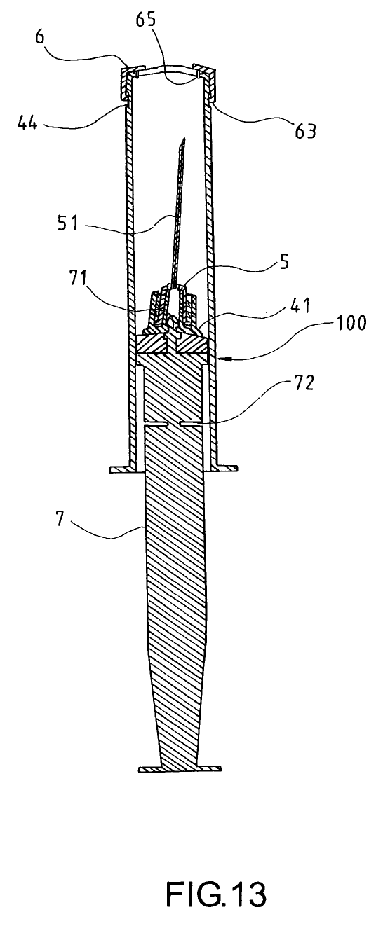

Referring to FIGS. 11, 12, and 13, the plunger 7 is continuously pushed forward after the injection has been completely made, such that the fastener 71 enters the opening 431 of the sheet 43 into the barrel 4 and passes through the opening 431, and the fastener 71 is hooked onto the sheet 43. In order to facilitate the fastening, the opening 431 of the sheet 43 configured is narrow shaped on the top and wide on the bottom. Therefore, the fastener 71 can easily enter and penetrate into the opening 431. Moreover, after the fastener 71 has been hooked, the protective cover 6 is turned downward thereby enabling the cutter 65 on its base thereof moving along the circular groove 411 of the barrel 4, and then cutting off the circular groove 411 when gradually moving downward. This way, the end portion 41 of the barrel 4 is separated from the housing of the barrel 4, and then the plunger 7 can be pulled back, such that the needle seat 5, the needle head 51, and the broken end portion 41 retract to the barrel 4 therein. As the slanted step portion 711 is disposed on the bottom rim of the fastener 71, the needle seat 5 and the needle head 51 are slanted thereby preventing the needle head from being pushed back, recycled and reused, and thus preventing patients from being infected by the needle head by completely damaging the barrel.

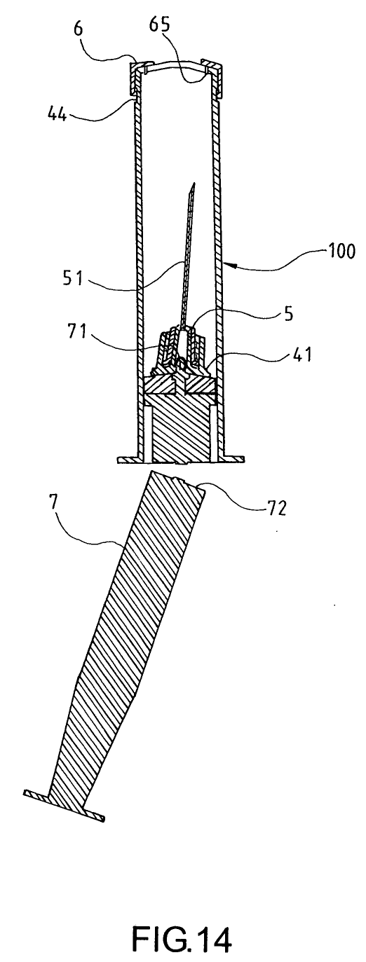

Referring to FIG. 14, after the plunger 7 has been pulled back for a certain length, it is possible to break it from the rectangular hole 72 along the edge of the opening of the barrel 4, so that the syringe 100 is completely damaged.

In summary, the present invention comprises the barrel having the broken portion, together with the plunger having a fastener and the structural design of the protective cover. Therefore, after the syringe has been used for injection, the needle head is retracted to the barrel therein, in order to completely damage the barrel, prevent it from being recycled and reused, and lower the production costs and defective rate of the safety syringe. As its pressure resistance, pull resistance, and retraction force have been confirmed to reach standard values upon testing, the present invention is deemed a practical, novel design.

It is of course to be understood that the embodiment described herein is merely illustrative of the principles of the invention and that a wide variety of modifications thereto may be effected by persons skilled in the art without departing from the spirit and scope of the invention as set forth in the following claims.

What is claimed is:

1. A disposable safety syringe structure comprising:
a barrel, an opening is formed in a center of a bottom rim of an outlet at an end portion thereof; a broken portion is disposed on a periphery of the end portion thereof; an annular groove is circumferentially formed on the end portion adjacent to a vertical surface thereof; a screw thread is disposed on a top of the annular groove thereof;
a protective cover, in shape of a hat, wherein a pore is formed in a center thereof for the needle seat to pass through; then a screw thread is disposed on an internal rim of a side thereof, to turn into the end portion of the barrel; a fastener is disposed on an internal edge of a bottom rim at the side thereof, to hook onto the annular groove of the barrel; a cutter arranged in intervals and corresponding to the broken portion of the barrel is disposed on a bottom thereof;
a plunger, disposed in the barrel therein; a vertical fastener is disposed in a center on a top thereof;
by means of the above structures, after an injection has been made, the plunger can be hooked onto the opening at the end portion of the barrel by a fastener, such that the protective cover is first turned, thereby making the cutter cutting off the broken portion of the barrel, and then pulling back the plunger, enabling the needle seat and the needle head retracting into the barrel.

2. The disposable safety syringe structure as claimed in claim 1, wherein a sheet is disposed on an internal edge of a bottom of the outlet in the end portion of the barrel; an opening is formed in the central of the sheet, thereby hooking the plunger onto the opening of the sheet by the fastener, after an injection has been made.

3. The disposable safety syringe structure as claimed in claim 1, wherein the broken portion is a circular groove in shape of a thin sheet formed in a periphery at the end portion of the barrel.

4. The disposable safety syringe structure as claimed in claim 1, wherein a slanted step portion is disposed on a bottom rim of the vertical fastener formed on a top end of the plunger, thereby enabling the needle seat and the needle head becoming slanted when being retracted, preventing the syringe from being reused or from causing infection.
